(19) United States
(12) Patent Application Publication
(10) Pub. No.: US 2018/0286506 A1
(43) Pub. Date: Oct. 4, 2018

(54) REMOTE CONTROLLED DIGITAL MEDICAL PRESCRIPTION PROCESSING SYSTEM

(71) Applicant: Ali Mahmoud Farhat, Dearborn, MI (US)

(72) Inventor: Ali Mahmoud Farhat, Dearborn, MI (US)

(21) Appl. No.: 15/958,650

(22) Filed: Apr. 20, 2018

Related U.S. Application Data
(63) Continuation of application No. 15/599,951, filed on May 19, 2017.
Application No. 15/599,951 is a continuation-in-part of application No. 15/472,344, filed on Mar. 29, 2017.
Application No. 15/472,344 is a continuation-in-part of application No. 15/472,344, filed on Mar. 29, 2017.

(51) Int. Cl. G16H 20/10; G06Q 50/22; G06Q 30/06; G16H 20/17; G16H 80/00; G16H 15/00; G06Q 20/08

(52) U.S. Cl. CPC G16H 20/10; G06Q 50/22; G06Q 30/0643; G06Q 20/085; G16H 20/17; G16H 80/00; G16H 15/00; G06Q 30/0603

(57) ABSTRACT

A pharmaceutical ordering and management system for providing an informational processing interface at a mobile device includes a mobile device including an interface configured to (1) present a display screen to receive user input to control a central processing unit that is operably coupled to at least one pharmacy; (2) transmit, via a wireless network, a mobile device command indicating an order, the command being generated in response to user input, (3) receive, via the wireless network and in response to the mobile device command, first data indicative of order status of the remote central processing unit, and; (4) present an updated display screen, the updated display screen reflecting the order information and history if any as a result of the mobile device command to present a real-time display of the pharmaceutical order status from the remote central processing unit.

### CROSS-REFERENCE TO RELATED APPLICATIONS

This application is a continuation of U.S. application Ser. No. 15/599,951, filed on May 19, 2017, which is a continuation-in-part of U.S. application Ser. No. 15/472,344 filed on Mar. 29, 2017, the disclosures of which are incorporated in their entireties by reference herein. This application is also a continuation-in-part of U.S. application Ser. No. 15/472,344 filed on Mar. 29, 2017.

### TECHNICAL FIELD

Embodiments disclosed herein generally relate to a remote controlled digital medical prescription processing system.

### BACKGROUND

Medical prescription processing systems have been used for cost-efficient, time-saving ways to store prescription needs, find pharmacies to fulfill those needs, place orders and arrange for pickup or delivery of the filled orders.

Among the art considered before filing this application are these references:

Known systems include GoodRX, but that system concentrates on coupons for patients.

### SUMMARY

One aspect of the disclosure is a system including an app that makes it easy for patients to order medicines using remote devices like smartphones, iPads, or desktop or laptop computers (collectively, “remote devices”). Such devices preferably include a memory that stores textual and graphical information. Textual information may for example include details about blood work, prescription needs, and medical records. Graphical images may for example include x-rays. Other medical test results can be downloaded from a central processor of the system to the remote device for future reference. A patient will have ready access to all previous results and prior medications taken before and after visiting a doctor.

In one embodiment, a patient will have options on the app to permit other users, such as other family members. Optionally, the user may create a record stored on the remote device for a pet and populate the record that details the pet's needs for medications.

A central processor communicates with the remote device wirelessly. Patient or a physician will upload a prescription need to the central processor. Optionally a patient may place an order for off-the-shelf medicines. Also, the patient may ask for home delivery or pickup at a pharmacy.

If desired, the app will remind patients of upcoming refills.

In practice, a user will create an account at his remote device. To do this, a picture ID and insurance information for the primary user may be required. Details of other family members may be supplied to permit their enrollment. Account information is uploaded to the central processor either directly or via a remote processor.

The central processor houses information about the inventories of local pharmacies and medical supply companies (collectively “retail outlets”), together with pricing information and whether the retail outlet has a relationship with various insurance carriers. If so, the central processor will capture information about the relevant deductibles and co-pays.

Via the central processor, the user may place an order for a prescription to be filled or refilled. The central processor will survey its database of local pharmacies, their inventory on hand, pricing and insurance companies with which they have a relationship. That information is summarized and communicated to the user via his remote device. Preferably, the information is presented in a sequence that lists pharmacies by location. Optionally the information could be sequenced by price. Armed with such information the user may then decide which pharmacy is best suited to fulfill the prescription need. He then places his order via his remote device to the central processor, which in turn communicates the order to the selected pharmacy.

Preferably the pharmacy acknowledges receipt of the order and communicates that fact to the user via the central processor together with a suggested pickup or delivery time.

Optionally, payment arrangements can be made directly from the user to the pharmacy via the central processor using a credit card on file or PayPal®. To do this, all participants will have a credit card on file with the central processor or pharmacy.

### BRIEF DESCRIPTION OF THE DRAWINGS

The embodiments of the present disclosure are pointed out with particularity in the appended claims. However, other features of the various embodiments will become more apparent and will be best understood by referring to the following detailed description in conjunction with the accompanying drawings in which:

FIG. 1 illustrate an example of initialization and setup including interactions between a user, a remote device and a central processing unit in accordance with one embodiment;

FIG. 2 illustrates an example transactions between the central processing unit and an insurance company in accordance with one embodiment;

FIG. 3 illustrates an example transaction between the central processing unit and a pharmacy in accordance with one embodiment;

FIG. 4 illustrates an example transaction between the user via the central processing unit and a pharmacy to place an order and delivery or pick-up instructions in accordance with one embodiment;

FIG. 5 illustrates an example series of transactions in which the insurance company pays the pharmacy and the user receives confirmation of the order details.

### DETAILED DESCRIPTION

As required, detailed embodiments of the present invention are disclosed herein; however, it is to be understood that the disclosed embodiments are merely examples of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

One aspect of the disclosure is a system such as an app that makes it easy for patients to order medicines using remote devices like smartphones or iPads or laptops or desktop computers (collectively, “remote devices”). Such devices preferably include a memory that stores textual and graphical information, Textual information may for example include details about blood work and other information typically housed in a medical record. Graphical images may for example include x-rays. Other medical test results can be downloaded on the system for future reference. A patient will have ready access to all results and prior medications taken before and after visiting a doctor.

The patient will have options on the app to permit other users, such as other family members. Optionally, the user may a create and populate one or more records stored on the remote device that detail needs for medications. In that context, as used herein the term “pharmacy” includes a veterinary or pet supply facility

A central processor communicates with the remote device wirelessly. Patients will upload a prescription need to the central processor. Optionally a patient may place an order for off-the-shelf medicines. Also, the patient may ask for home delivery or pickup at a pharmacy.

If desired, the app will remind patients of upcoming refills.

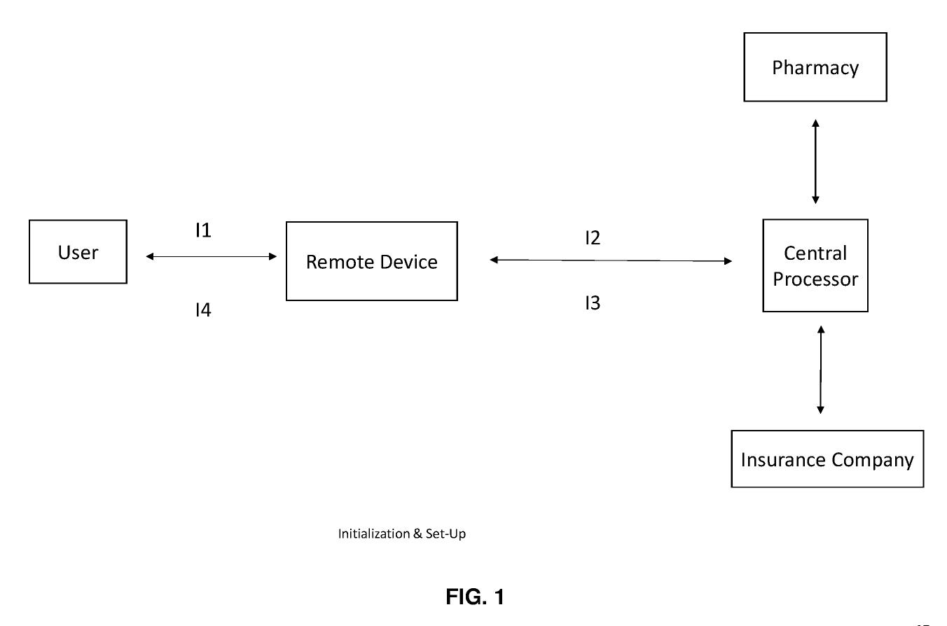

In practice, as shown in FIG. 1 a user will create an account at his remote device (transaction I1). To do this, a picture ID and insurance information for the primary user may be required. Details of other family members may be supplied to permit their enrollment. Account information is then uploaded to a central processor (transaction I2)

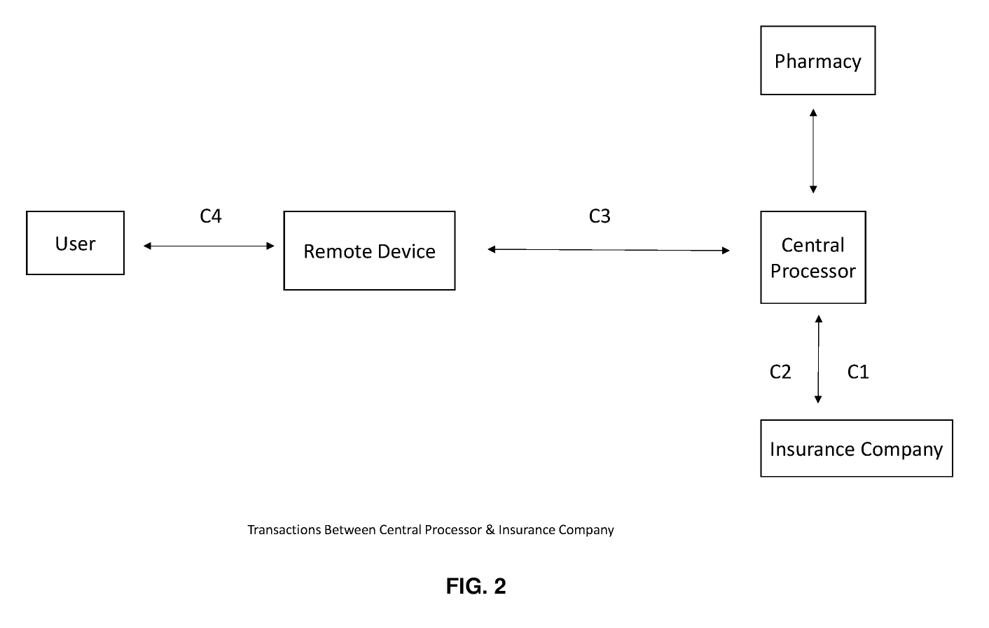

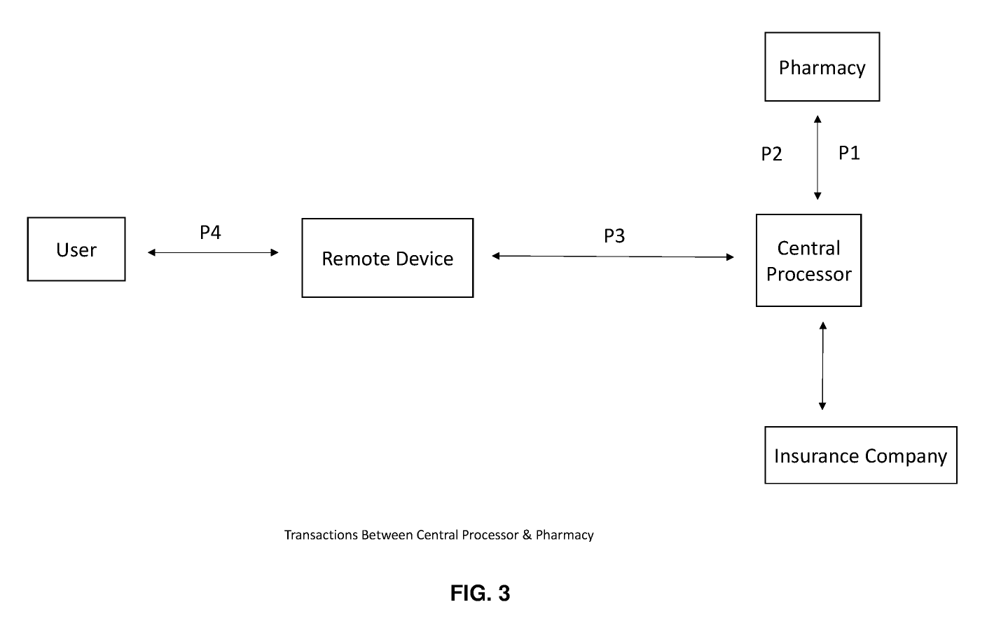

The central processor houses information about the inventories of local pharmacies (FIGS. 2, 3) and medical supply companies (collectively “pharmacies” or “retail outlets”), together with pricing information and whether the retail outlet has a relationship with various insurance carriers (transactions C1, C2, C3, C4, P1, P2, P3, P4) If so, the central processor will capture information about the relevant deductibles and co-pays and communicate that information to the user via the remote device (transactions C3, C4).

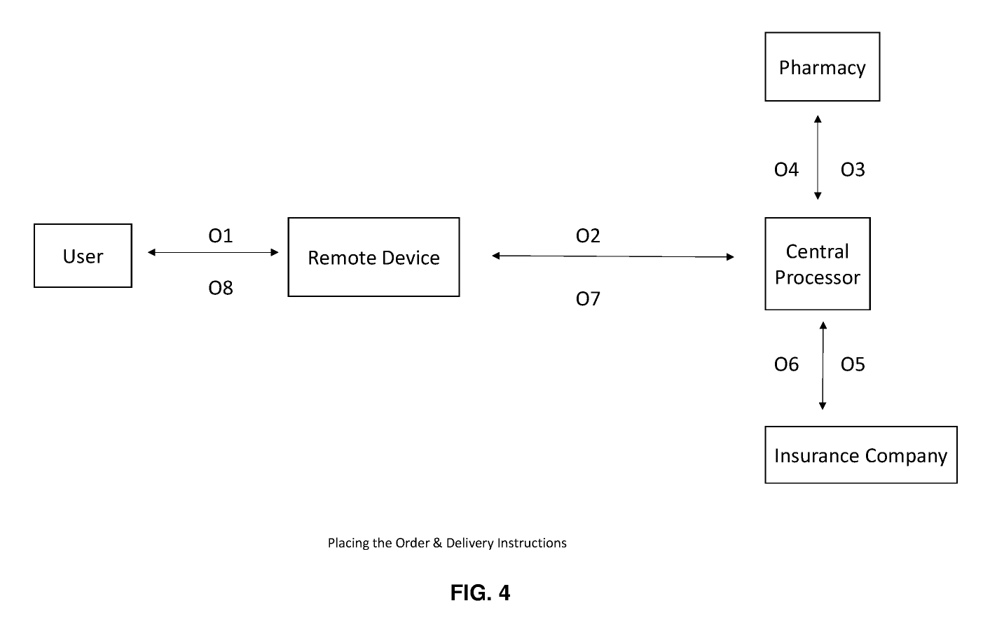

Via the central processor, the user may place an order for a prescription to be filled or refilled (FIG. 4, transactions O1, O2). The central processor will survey its database of local pharmacies, their inventory on hand, and pricing and insurance companies (transactions O5, O6) with which they have a relationship. That information is summarized and communicated to the user via his remote device (transactions O7, O8). Preferably, the information is presented in a sequence that lists pharmacies by location. Optionally the information could be sequenced by price. Armed with such information the user may then decide which pharmacy is best suited to fulfill the prescription need. He then places his order via his remote device to the central processor, which in turn communicates the order to the selected pharmacy (transactions O3, O4).

Preferably the pharmacy acknowledges receipt of the order (transaction O4, O7, O8) and communicates that fact to the user via the central processor together with a suggested pickup or delivery time.

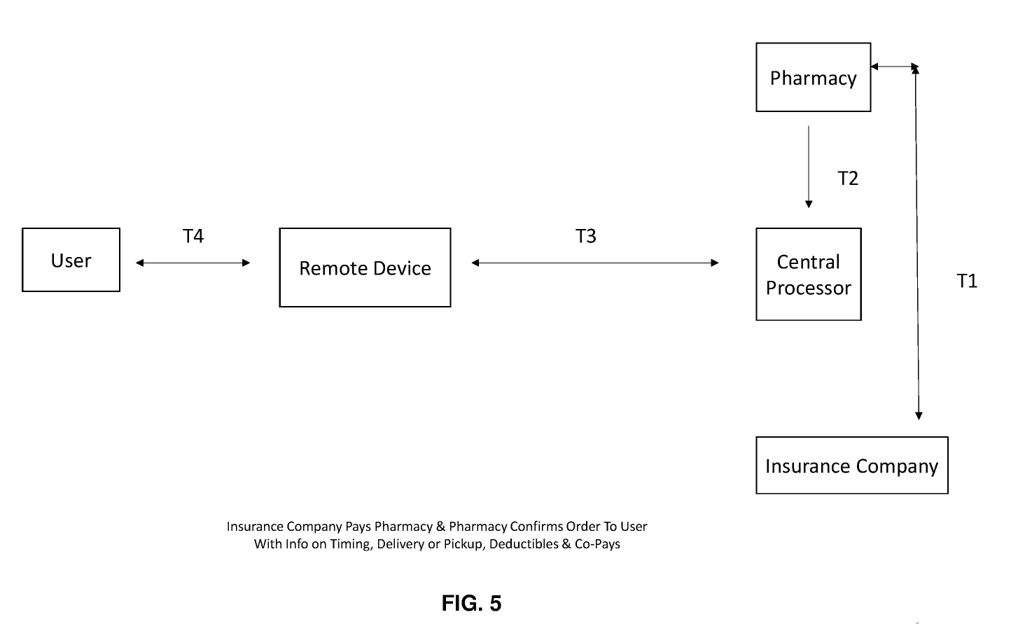

Optionally, payment arrangements can be made by the insurance company (transaction T1, FIG. 5), directly from the user to the pharmacy via the central processor using a credit card on file or PayPal® or other payment means. To do this, all participants will have a credit card on file with the central processor or pharmacy. Upon payment, the pharmacy will confirm with the central processor (transaction T2), which in turn communicates order confirmation and pickup/delivery information to the user via the remote device (transactions T3, T4).

In the embodiments described herein, the remote device may include a keypad, a memory and a processor. The remote device may be portable, or be a desktop or laptop computer. The remote device may include a built-in router and be capable of wireless communication with a central processor. Users may access and control certain settings of the central processor via for example a hypertext markup language (“HTML”) 5 user interface using a web browser onboard the mobile device. Thus, since a web browser may open the user interface, the central processor settings may be implemented on any mobile device, regardless of the type, brand, or operating system of the device. No additional device specific application is required. The mobile device may then communicate directly with the central processor and apply the desired settings via user interaction at the mobile device. The user interface at the mobile device may be configured to save settings, profiles, prescriptions, etc., which may be easily recalled and applied.

Furthermore, more than one mobile device may concurrently control the central processor. That is, multiple browser windows may operate concurrently and remain in synchronization with one another to control the central processor. For example, a single user may have a main pharmaceutical order page open on a laptop computer while having a page open on a tablet for editing. When the user selects a pharmacy on the laptop, optionally a page may be available for editing on the tablet. Thus, the pharmacy interaction function may follow across multiple browsers and devices. The system may also facilitate unlimited user configurable identifications and allow multiple users to set up their own unique way of communicating with the central processor or pharmacy.

Accordingly, the central processor may be remotely managed with any type of device, even one not used with the central processor previously. These capabilities create an easy, portable pharmaceutical communications system that may be set-up quickly and accessed easily without the need for applications on a specific device.

FIGS. 1-5 illustrate for example one central processing unit, of which there may be one or more, (collectively referred to herein as a central processing unit) in accordance with one embodiment. The central processing unit may have wireless communication capabilities, as described herein. The central processing unit may include an Ethernet™ connection, additional USB ports and a power source connection, and a cascade connector (e.g., cat-5 connector) to facilitate the cascading of multiple central processing units.

The central processing unit may also include an antenna and a light emitting diode (LED) 160, which may be configured to illuminate to indicate a connection with a wireless network. Although not shown, the Ethernet™ connection, reset switch, footswitch, additional USB ports and HDMI output may be arranged on a side panel of the central processing unit.

The central processing unit may include at least one handle and may be configured to be portable and easily moved from one location to the next. The central processing unit may also be rack-mountable.

The system may also include any number of mobile devices. Each mobile device may include a wireless transceiver (e.g., a BLUETOOTH module, a ZIGBEE transceiver, a Wi-Fi transceiver, an IrDA transceiver, an RFID transceiver, etc.) configured to communicate with the central processing unit and/or a remote server.

Each mobile device may include a device display screen configured to display information to a user and to receive commands from the user. The interfaces displayed via the display screen may be any one of, or a combination of visual displays such as light emitting diodes (LEDs), organic LED (OLED), Active-Matrix Organic Light-Emitting Diode (AMOLED), liquid crystal displays (LCDs), thin film diode (TFD), cathode ray tube (CRT), plasma, a capacitive or resistive touchscreen, etc.

As shown in FIGS. 1-5, each mobile device is configured to communicate with the central processing unit. Each mobile device may be capable of accessing an HTML5 webpage (i.e., the device may be HTML5 compatible). The mobile device may communicate directly with the central processing unit via a wireless network (not shown). Further, each mobile device may be any device capable of handling HTTP regardless of the device's platform (i.e., any device including iOS®, Android®, Windows®, Mac® OS, Linux®, etc., platforms). The central processing unit, as explained, may be capable of wireless communication without the use an external router. Thus, additional hardware and set-up thereof is not necessary to enable wireless communication with the central processing unit.

Each remote server or central processor may store and transmit updates (e.g., additional signal processing algorithms) to each mobile device.

As shown, the central processing unit may include a wireless access point and a web server configured to process requests via, by example, HTML, specifically HTML5. The wireless access point may facilitate a connection to the wireless network. Thus, commands may be sent from the mobile device directly to the central processing unit without the need for an external router. The web server may include Hyper Text Markup Language (HTML) 5, websockets to open an interactive communication session between the user's browser and a server. By using a websocket based application program interface (API), messages may be transmitted and received without having to poll an external server for a reply.

In use, once a user at the mobile device opens an app or a browser and enters the appropriate uniform resource identifier (URL), the user may enter his or her credentials (e.g., user name and password) or allow identification via a finger print. The mobile device uses such a user input to transmit a command over a socket connection to the web server of the central processing unit. The socket connection facilitates communication between the mobile device and the web serve. Once this bi-directional communication is open, the central processing unit may receive commands from the mobile device. Concurrently, the web server may send feedback to the mobile device 115 and the user interface at the mobile device may be updated accordingly.

The central processor may communicate with multiple mobile devices at a time. When multiple mobile devices are simultaneously transmitting commands to the central processor, each mobile device may, in real-time or near real-time, display the effects of the commands sent by another mobile device. For example, if a first user at a first one of the mobile devices transmits an order, a second user at a second one of the mobile devices may then see the order placed by the first user at the second mobile device. In addition to processing being recognized nearly simultaneously across multiple mobile devices, each mobile device operates independently of the other.

It will be appreciated that the central processing unit may include a processor and a one or more databases configured to perform instructions, commands and other routines in support of the processes described herein. For example, the central processor may be configured to execute signals generated by commands entered by a user at a remote device to generate signals(s) to provide an input to a pharmacy. The processor may include a controller (not shown) and may include a dual-core processor (e.g., an ARM® processor) configured to interface with the web server and perform other signal processing if needed on-board the central processing unit.

The database may save historical transaction records placed by one or more users. These records may be user specific and the database may maintain user profiles and settings associated therewith. Groups and sub-groups, including settings and security information therefor, may be maintained in the database. The database may receive updates from the mobile device, including software updates, as well as updated user information (including user profile updates and settings).

The mobile device may access and communicate with the central processing unit identification such as an internet protocol (IP) address of the central processing unit. Once the mobile device accesses the web server via the websocket, various user settings, presets, etc., may be adjusted in the HTML5 interface. Certain settings may be saved and recalled for later use. Additionally, certain security settings may be included via username/password combinations that are enterable at the HTML5 interface. In these settings, specific access may be given to certain login combinations. For example, a first user, or administrator, may have access to change credit card or other payment information and adjust, change, save, etc., any and all settings for all of the users that are operably connected to the central processing unit. In another example, another user may only have access to the settings as they relate to his or her order.

Not shown are example screens for the user interface to be displayed via the web browser on the mobile device. The interfaces include various features configured to control the central processing unit in response to user inputs at the display screen of the mobile device. Shortcuts to various features and settings may be included throughout the interface to increase usability and provide a better user experience. The various interfaces may facilitate certain navigation and user gesture techniques to create a user-friendly interface system.

Because the central processing unit may be controlled from any number of devices, the central processing unit may continually send updated data back to the mobile devices. As a user navigates through the interfaces on the mobile device, the screens will be updated in real-time or near real-time. Upon receiving user input that is indicative of a change, a command may be transmitted via the wireless communication between the mobile device and the central processing unit indicating the change. The central processing unit, upon receiving the command, may apply the change and if appropriate transmit the updated data back to the mobile device so that the corresponding screens reflect the change. The updated data is also transmitted to any other mobile device currently communicating with the central processing unit.

In one example case (FIG. 4, transactions O1-O8), the central processing unit may receive a mobile device command from the mobile device. The mobile device command may include for example an order for a particular medicine. The mobile device command may be received from any number of mobile devices. For example, the mobile device command may transmitted by a laptop and another command may be transmitted by a mobile phone.

In response, the central processing unit may transmit a confirmation to the mobile device. Thus, each time a command is entered at the mobile device, updated signals are transmitted back to the mobile device so that the interfaces thereon reflect the current state of the central processing unit. That is, the display screen may be continually updated. Furthermore, regardless of which mobile device transmitted the mobile device command, each mobile device in communication with the central processing unit may receive the updated data and therefore be configured to display the most up to date interfaces.

Such processes may proceed until the central processing unit is powered down, or until each of the mobile devices is no longer communicating with the central processing unit.

In one example the mobile device transmits various mobile device commands and displays in real-time or near real-time the state of the central processing unit. The user input may include opening a web browser on the device and entering user credentials such as a user-name and password.

After the user is authenticated via the remote server or web server, the mobile device may transmit, via a wireless network, a request or order to the central processing unit. The mobile device request may include a request to create a connection with the central processing unit.

In response to the request, the mobile device may receive confirmation of the request or order from the central processor.

Accordingly, the central processing unit is a complete pharmaceutical order placement and processing system that is compact and portable with the ability to be controlled by any connected device via a standard web browser without the need for device specific applications, and without operating system limitations. The central processing unit is simple and secure, allowing the user to easily handle his or her needs for management of pharmaceutical needs. The web-based mobile device controlled system may allow users to control the central processing unit and its settings remotely over a wireless network.

Computing devices if present, such as the central processing unit, remote device, external server, remote server etc., generally include computer-executable instructions, that may be executable by one or more computing devices such as those listed. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of computer-readable media.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A non-transitory computer-readable medium tangibly embodying computer-executable instructions of a software program, the software program being executable by a processor of a computing device to provide operations, comprising:
presenting, via a web browser on a mobile device, a display screen to receive user input to control a central processing unit that is operably coupled to at least one pharmacy;
transmitting, via a wireless network, a command from the mobile device, the command indicating an order for a pharmaceutical, the mobile device command being generated in response to user input at the display screen of the mobile device;
receiving by the central processing unit, via the wireless network and in response to the mobile device command, the pharmaceutical order; and
presenting an updated display screen on the mobile device, the updated display screen reflecting the order as a result of the mobile device command to present a real-time display of the order status.

2. The medium of claim 1, further comprising:
receiving by a pharmacy, via the wireless network, an order for a pharmaceutical or off-the-shelf item from the remote central processing unit.

3. The medium of claim 2, wherein an updated display screen of the remote device includes indicia that signify confirmation of the status of the order.

4. The medium of claim 3, wherein the user input includes a selection of the pharmaceutical or off-the-shelf item to be ordered.

5. The medium of claim 4, further comprising presenting at least one screen specific to the central processing unit channel corresponding to a selected order.

6. The medium of claim 1, wherein the updated display screen includes a confirmation of an order having been placed.

7. The medium of claim 6, wherein the updated display screen includes payment information.

8. The medium of claim 6, wherein the updated display screen is updated in real-time based on one or more signals from the central processing unit.

9. A pharmaceutical ordering system for providing an order processing interface at a mobile device, comprising:
a mobile device including an interface configured to:
present a display screen to receive user input to control a central processing unit that is operably coupled to at least one pharmacy;
transmit, via a wireless network, a mobile device command indicating an order for a pharmaceutical, the command being generated in response to user input;
receive, via the wireless network and in response to the mobile device command, a signal indicative of order status from the remote central processing unit; and
present an updated display screen, the updated display screen reflecting the order status as a result of the mobile device command to present a real-time display of the order status of the remote central processing unit.

10. A pharmaceutical ordering system including an app that makes it easy for patients to order medicines using remote devices like smartphones, iPads, or desktop or laptop computers (collectively, “remote devices”), the system including;
one or more of the remote devices including a memory that stores textual and graphical information;
the textual information including details about blood work, prescription needs, and medical records;
the graphical information if present including one or more images such as x-rays and other medical test results that can be downloaded from a central processor to the remote device, thereby affording to the patient access to at least some previous results and prior medications taken before and after visiting a doctor.

11. The pharmaceutical ordering system of claim 10, wherein the app further includes means for providing a patient with an option to permit other users, such as other family members.

12. The pharmaceutical ordering system of claim 10, wherein the app further includes means for permitting the user to create a record stored on the remote device for a pet and populate the record with details of the pet's needs for medications.

13. The pharmaceutical ordering system of claim 10, further including a central processor that communicates with the remote device wirelessly so that a patient or a physician can upload a prescription need to the central processor or an order for off-the-shelf medicines while specifying home delivery or pickup at a pharmacy.

14. The pharmaceutical ordering system of claim 10, wherein the app reminds patients of upcoming refills.

15. The pharmaceutical ordering system of claim 10, wherein the app permits a user to create an account at a remote device using a picture ID and insurance information for the primary user, the account including details of other family members for enrollment, the account information being uploaded to the central processor.

16. The pharmaceutical ordering system of claim 15, wherein the central processor houses information about the inventories of local pharmacies and medical supply companies (collectively “retail outlets”), together with pricing information and whether the retail outlet has a relationship with various insurance carriers, the central processor capturing information about relevant deductibles and co-pays.

17. The pharmaceutical ordering system of claim 16, wherein
a. the user places an order for a prescription to be filled or refilled;
b. the central processor surveys its database of local pharmacies, their inventory on hand, pricing and insurance companies with which they have a relationship and communicates to the user via his remote device;
c. the user decides which pharmacy is best suited to fulfill the prescription need and places his order via his remote device to the central processor, which in turn communicates the order to the selected pharmacy.

18. The pharmaceutical ordering system of claim 17, wherein
a. the pharmacy acknowledges receipt of the order and communicates that fact to the user via the central processor together with a suggested pickup or delivery time.

19. The pharmaceutical ordering system of claim 18, wherein
a. payment arrangements are made directly from the user to the pharmacy via the central processor using a credit card on file or PayPal®, the user having a credit card on file with the central processor or pharmacy.
